(19) United States
(12) Patent Application Publication
(10) Pub. No.: US 2004/0219586 A1
(43) Pub. Date: Nov. 4, 2004

(54) Unit for biochemical analysis

(75) Inventors: Koji Kuruma, Minami-ashigara-shi (JP); Hiroko Inomata, Asaka-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/808,451

(22) Filed: Mar. 25, 2004

(30) Foreign Application Priority Data
Mar. 28, 2003 (JP) 2003-090369

(51) Int. Cl. C12Q 1/68; G01N 33/53; C12M 1/34

(52) U.S. Cl. 435/6; 435/7.1; 435/287.2

(57) ABSTRACT

The present invention provides a unit for biochemical analysis wherein the unit comprises a substrate formed of a material having properties of attenuating radiation and/or light and formed with a plurality of holes, and adsorptive areas are respectively formed inside the plurality of holes, thereby forming a plurality of adsorptive areas, and wherein covalently binding functional groups are introduced onto the adsorptive areas. The present invention enables to provide a unit for biochemical analysis which is capable of carrying out strong and efficient immobilization of specific binding substances and can obtain specific and high signals by controlling the direction of the immobilized specific binding substances.

### TECHNICAL FIELD

The present invention relates to a unit for biochemical analysis comprising adsorptive areas to which a functional group is introduced, and a method for conducting biochemical analysis using the same. More specifically, the present invention relates to a unit for biochemical analysis comprising a plurality of adsorptive areas formed separately from each other which areas are capable of immobilizing specific binding substances (for example, ligand, receptor, etc.) via a covalent bond, a method for producing the same, a method for immobilizing a specific binding substance using the same, and a method for biochemical analysis using the same.

### BACKGROUND ART

In recent years, there have been developed microarray analysis systems wherein a spotter equipment is used to spot hormones, tumor markers, enzymes, antibodies, antigens, abzymes, other proteins, nucleic acids, cDNAs, DNAs, RNAs and other specific binding substances which can specifically bind to a substance derived from a living organism and their nucleotide sequences or their nucleotide length and composition, etc. are known at different positions on a support surface such as a slide glass and a membrane filter so as to form a large number of independent spots; subsequently a substance such as hormones, tumor markers, enzymes, antibodies, antigens, abzymes, other proteins, nucleic acids, cDNAs, DNAs, mRNAs and other substances obtained from a living organism by extraction, isolation or the like and optionally subjected to treatment such as chemical treatment and chemical modification and labeled with labeling substances such as a fluorescent substance and dye, is allowed to be specifically bound to the specific binding substances by hybridization, etc.; an exciting light is irradiated to this microarray, and the light such as fluorescence emitted from the labeling substance such as the fluorescent substance and the dye is photoelectrically detected, thereby the substance from the living organism is analyzed. According to this microarray analysis system, since a large number of spots of specific binding substances are formed in high density at different positions on a support surface such as a slide glass and a membrane filter and hybridized with a substance derived from a living organism and labeled with labeling substances, there is an advantage that analysis of the substance from the living organism can be effected in a short time.

There have been also developed macroarray analysis systems using a radioactive labeling substances wherein a spotter equipment is used to spot hormones, tumor markers, enzymes, antibodies, antigens, abzymes, other proteins, nucleic acids, cDNAs, DNAs, RNAs and other specific binding substances which can specifically bind to a substance derived from a living organism and their nucleotide sequences or the nucleotide length and composition of bases; etc. are known at different positions on a support surface such as a membrane filter to form a large number of independent spots; subsequently a substance such as hormones, tumor markers, enzymes, antibodies, antigens, abzymes, other proteins, nucleic acids, cDNAs, DNAs, mRNAs and other substances obtained from a living organism by extraction, isolation or the like and optionally subjected to treatment such as chemical treatment and chemical modification and labeled with a radioactive labeling substances is allowed to be specifically bound to the specific binding substances by hybridization, etc.; this macroarray is closely contacted with an accumulative fluorescent substance sheet on which a photostimulable phosphor layer containing a photostimulable phosphor is formed; the photostimulable phosphor layer is exposed to light; and after that an exciting light is irradiated to the photostimulable phosphor layer; and the photostimulated light emitted from the photostimulable phosphor layer is photoelectrically detected to generate data for biochemical analysis, thereby the substance from the living organism is analyzed.

The units conventionally used for biochemical analysis commonly utilize a method of non-covalently immobilizing the specific binding substance. The specific binding substance to be immobilized may be immobilized by post-treatment such as UV irradiation, when the substance is a nucleic acid such as DNA. Each method has difficulties in the control of the direction and binding site of the specific binding substance to be immobilized.

The system in which the substance is covalently immobilized highly probably causes decrease in binding ability, because, as is particularly remarkable in the case of a nucleic acid, especially a short chain DNA such as a synthetic oligonucleotide, a part of bases in the oligonucleotide is generally used for immobilization and the bases capable of binding the target substance decreases. Furthermore, immobilization ratio by non-covalent binding is low in the case of a short chain DNA such as a synthetic oligonucleotide, where significant amount of oligonucleotides will exfoliate, although the amount of immobilization can be fully maintained, for example, in the case of a long chain DNA. This will also cause significant decrease in sensitivity. When the ligand or receptor to be immobilized is a protein, they are commonly bound non-covalently by electrostatic bond and hydrophobic bond. In this case, it is not only difficult to define the part of the protein binding to the adsorptive area but also highly probable to cause denaturation of the protein.

JP Patent Publication (Kokai) No. 2002-355036A discloses a unit for biochemical analysis characterized in that the unit comprises a substrate formed of a material having properties of attenuating radiation and/or light and formed with a plurality of holes and that adsorptive areas are respectively formed inside the above-mentioned a plurality of holes thereby forming a plurality of adsorptive areas, and a method for biochemical analysis using the unit.

International Patent Publication WO 00/34457 discloses a method for immobilizing an oligonucleotide on the support by spotting a buffer solution containing the oligonucleotide on the support such as glass, characterized in that the oligonucleotide is immobilized on the support via a covalent bond.

JP Patent Publication (Kokai) No. 5-168499A (1993) discloses an oligonucleotide probe reagent containing a nylon film with having anionic carboxyl groups in high density on which at least one of oligonucleotide probes including 5′-amine is covalently bonded via amide bond, and its manufacturing method.

### DISCLOSURE OF THE INVENTION

An object to be achieved by the present invention is to eliminate the above-mentioned problems of the conventional art. That is, an object to be achieved by the present invention is to provide a unit for biochemical analysis which is capable of carrying out strong and efficient immobilization of specific binding substances and wherein specific and high signals can be obtained by controlling the direction of the immobilized specific binding substances. Further object to be achieved by the present invention is to provide a method for biochemical analysis using the above-mentioned unit for biochemical analysis, a production method of the above-mentioned unit for biochemical analysis, and a method for immobilizing the specific binding substance using the above-mentioned unit for biochemical analysis.

The present inventors have conducted intensive studies to achieve the above-mentioned objects and have found that, as to the unit for biochemical analysis which comprises a substrate formed of a material having properties of attenuating radiation and/or light and formed with a plurality of holes and wherein adsorptive areas are respectively formed inside the above-mentioned a plurality of holes thereby forming a plurality of adsorptive areas, a unit for biochemical analysis which exhibits a desired effect can be provided by introducing a covalently binding functional group onto the adsorptive areas. The present invention has been completed based on this finding.

Thus, the present invention provides a unit for biochemical analysis wherein the unit comprises a substrate formed of a material having properties of attenuating radiation and/or light and formed with a plurality of holes, and adsorptive areas are respectively formed inside the plurality of holes, thereby forming a plurality of adsorptive areas, and wherein covalently binding functional groups are introduced onto the adsorptive areas.

Another aspect of the present invention provides a unit for biochemical analysis wherein the unit comprises an adsorptive substrate formed of an adsorptive material having covalently binding functional groups and a perforated plate formed with a plurality of through-holes and formed of a material having properties of attenuating radiation and/or light, said perforated plate being closely contacted with at least one surface of said adsorptive substrate to form a plurality of adsorptive areas of said adsorptive substrate in said plurality of through-holes formed in said perforated plate.

Still another aspect of the present invention provides a unit for biochemical analysis wherein the unit comprises a substrate formed of a material having properties of attenuating radiation and/or light and formed with a plurality of holes, and adsorptive areas are respectively formed inside the plurality of holes thereby forming a plurality of adsorptive areas, and wherein a specific binding substance whose structure or characteristics is known is covalently bound on the adsorptive areas and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with said specific binding substance so that said plurality of adsorptive are selectively labeled.

Preferably, the specific binding substance whose structure or characteristics is known has a functional group.

Preferably, the specific binding substance having a functional group is selected from a group consisting of nucleic acids, proteins and peptides.

Preferably, the nucleic acids having a functional group are selected from a group consisting of nucleotide derivatives, peptide nucleic acids and LNA.

Preferably, the nucleotide derivatives having a functional group are oligonucleotides.

Preferably, the substance derived from a living organism is bound with said specific binding substance by a reaction selected from a group consisting of hybridization, antigen-antibody reaction and receptor-ligand reaction.

Preferably, the adsorptive areas hold the covalently binding functional groups via a spacer.

Still another aspect of the present invention provides a method for biochemical analysis wherein the unit for biochemical analysis according to the present invention is used, and wherein a specific binding substance whose structure or characteristics is known is covalently immobilized on the adsorptive areas of the unit for biochemical analysis, and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with the specific binding substance thereby detecting said labeled substance derived from a living organism.

Preferably, the substance derived from a living organism is specifically bound with said specific binding substance by a reaction selected from a group consisting of hybridization, antigen-antibody reaction and receptor-ligand reaction.

Still another aspect of the present invention provides a method for producing a unit for biochemical analysis wherein the unit comprises a substrate formed of a material having properties of attenuating radiation and/or light and formed with a plurality of holes and adsorptive areas are respectively formed inside the plurality of holes thereby forming a plurality of adsorptive areas, which comprising a step of closely contacting a material having a covalently binding functional group with the substrate.

Still another aspect of the present invention provides a method for manufacturing a unit for biochemical analysis wherein the unit comprises a substrate formed of a material having properties of attenuating radiation and/or light and formed with a plurality of holes and adsorptive areas are respectively formed inside the plurality of holes thereby forming a plurality of adsorptive areas, which comprises a step of introducing a covalently binding functional group into the adsorptive material closely contacted with the substrate.

Preferably, the adsorptive material is a porous material.

Still another aspect of the present invention provides a method for immobilizing a specific binding substance to the unit for biochemical analysis according to the present invention which comprises a step of treating the adsorptive area where a functional group is held with an activating agent for improving reactivity.

Preferably, after a step of treating the adsorptive area where a functional group is held with an activating agent for improving reactivity, a specific binding substance having a functional groups is reacted and immobilized.

Preferably, a spacer is held between the specific binding substances having a functional group and the adsorptive areas.

### BEST MODE FOR CARRYING OUT THE INVENTION

The mode for carrying out the present invention will be described hereafter.

The unit for biochemical analysis of the present invention comprises a substrate formed of a material having properties of attenuating radiation and/or light and formed with a plurality of holes, and adsorptive areas are respectively formed inside the above-mentioned a plurality of holes thereby forming a plurality of adsorptive areas. The unit for biochemical analysis of the present invention is characterized in that a covalently binding functional group is introduced onto the adsorptive areas.

A specific binding substance is bound via a covalent bond by introducing a covalently binding functional group which can bind with a plurality of adsorptive areas formed separately from each other in the unit for biochemical analysis. This attains immobilization stronger and more efficient than the conventional method. Furthermore, specific binding capability is characteristically exploited to the maximum extent by controlling the direction of a specific binding substance by biding the adsorptive area and the specific position of the specific binding substance. Consequently, sensitization much higher the conventional method can be achieved.

This method is effective particularly in the case that the specific binding substance to be immobilized is a short chain DNA such as a synthetic oligonucleotide which is hard to be immobilized. The method may be also extremely useful in the case of protein where denaturation at the time of immobilization is a significant problem.

As for the method for introducing a covalently binding functional group to the adsorptive area, either one of the methods that the unit for biochemical analysis is subjected to some treatment to introduce the functional group or that the unit for biochemical analysis is manufactured from an adsorptive material to which the functional groups have been introduced beforehand, can be used. Examples of the methods for post-treating the unit for biochemical analysis include a method of coating a polymer (synthetic polymer, natural polymer, etc.) having a functional group, a method of forming a polymer on the surface of the adsorptive area from a monomer having a functional group by plasma polymerization or graft polymerization, a treatment with a bifunctional low molecular compound which can bind with functional groups on the surface of the adsorptive material. Examples of the adsorptive materials to which the functional groups have been introduced beforehand include polymers or copolymers polymerized from a monomer having a functional group, polymers having an amino group and a carboxyl group at the end of molecules such as nylon, polysaccharides which have been reduced and imparted with a functional group, blended articles of these materials and commercial adsorptive materials having any functional groups (for example, Biodyne C, Immunodyne ABC, UltraBind, LoProdyne, etc. available from Pall Corporation), etc.

The term “specific binding substance” as used herein means “any member which forms a biologically specific bond”, and includes for example, receptor, ligand, etc.

In the present invention, the adsorptive area of the unit for biochemical analysis or the material from which the adsorptive area is formed is treated with a polymer compound having a covalently binding functional group, thereby a covalently binding functional group can be introduced. Examples of synthetic polymers having a covalently binding functional group include a homopolymer or copolymer obtained by using acrylic acid, methacrylic acid, acrylamide, methyl methacrylate, glycidyl methacrylate, allylamine, allyl aldehyde, vinyl acetic acid, etc. as a monomer, and polylysine, etc. Examples of the natural polymers include polysaccharides, alginic acid, polysaccharides aldehydated by periodic acid oxidization, aldehydated polysaccharide further carboxylated by sodium chlorite, protein such as collagen, gelatin and casein, etc.

The unit for biochemical analysis of the present invention can be produced from the adsorptive material having a covalently binding functional group. Adsorptive material may be a single substance of polymer compound having a covalently binding functional group, or its complex. Examples of synthesized or natural polymers having a covalently binding functional group include a homopolymer or copolymer obtained by using acrylic acid, methacrylic acid, acrylamide, methyl methacrylate, glycidyl methacrylate, allylamine, allyl aldehyde, vinyl acetic acid, etc. as a monomer, polylysine, polysaccharides such as alginic acid, polysaccharides aldehydated by periodic acid oxidization, aldehydated polysaccharide further carboxylated by sodium chlorite, protein such as collagen, gelatin and casein, etc. These single substances, or nylons such as nylon-6, nylon-6,6, nylon-4,10; cellulose derivatives such as nitrocellulose, cellulose acetate, cellulose butyrate acetate; collagen; alginic acids such as alginic acid, calcium alginate, alginate-polylysine polyionic complex; polyolefins such as polyethylene and polypropylene; polyvinyl chloride; polyvinylidene chloride; polyfluorides such as polyvinylidene fluoride and polytetrafluoride, and a complex with these copolymers can also be used.

In the present invention, the adsorptive area of the unit for biochemical analysis or the surface of a material from which the adsorptive area is formed may be made into a polymer having a covalently binding functional group by graft polymerization or plasma polymerization. As a monomer, acrylic acid, methacrylic acid, acrylics amide, methyl methacrylate, glycidyl methacrylate, allylamine, allyl aldehyde, vinyl acetic acid, etc. can be used.

In the present invention, the adsorptive area of the unit for biochemical analysis or the surface of a material from which the adsorptive area is formed can also be treated with a low molecular compound. Examples of low molecular compounds include triazine, vinyl sulfone, hydroxysuccinimide, maleimide, glutaraldehyde, etc.

For the binding of a specific binding substance and an adsorptive area, a reaction generally known as a condensation reaction or a crosslinking reaction can be used. For example, it can be conducted by crosslinking between amino groups by glutaraldehyde, covalent bonding between an amino group and a carboxyl group by carbodiimide alone or carbodiimide and NHS, insertion reaction by photodegradation of azide, exchanging reaction of an amino group and a tosyl group, a reaction between a thiol group and a maleimide group, a reaction between an azide group and an amino group, a reaction between an isocyanate group and a hydroxyl group, a reaction between an isothiocyanate group and an amino group, a reaction between an amino group, an imino group, a hydrazino group, a carbamoyl group, a hydrazinocarbonyl group, a carboxyimido group or a mercapto group and a vinylsulfonyl group, a reaction between a thiol group and a halogenated acetyl group, a reaction between a hydroxyl group and an epoxy group, a reaction via a Schiff base between an amino group and an aldehyde group, a reaction between an aldehyde group and a hydrazide group, etc.

In the present invention, a specific binding substance can be immobilized to the unit for biochemical analysis by a process which treats the adsorptive area where the functional group is held with an activating agent for improving reactivity. The activating agent for improving reactivity which can be used in the present invention refers to an activating agent used for activating a carboxyl group, an amino group, a thiol group, etc. which are “covalently binding functional groups”, and examples for COOH include 1-ethyl-3-(3-dimethylaminopropyl)carbodiimide hydrochloride (abbreviated as EDC according to the catalog of WAKO Pure Chemicals, Co.) (water-soluble carbodiimide) and NHS (N-hydroxysuccinimide), and examples for amino group include divinyl sulfone, glutaraldehyde and a bifunctional activating agent which reacts with both thiol group and amino group called as crosslinker. In the Examples given in this specification, Biodyne C (EDC+NHS for COOH-introduced membrane) and divinyl sulfone for nylon membrane (membrane having NH2) are used for activation.

The details of the unit for biochemical analysis of the present invention are described in JP Patent Publication (Kokai) No. 2002-355036A (2002), and all the contents given in JP Patent Publication (Kokai) No. 2002-355036A (2002) shall be incorporated into the present specification as a part of disclosure of the present specification. JP Patent Publication (Kokai) No. 2002-355036A (2002) specifically describes the units for biochemical analysis given in the following (1) to (29). The units for biochemical analysis similar to the following (1) to (29) provided that the adsorptive area or adsorptive material has a covalently binding functional group, can be used for the adsorptive area or adsorptive material in the present invention.

(1) A unit for biochemical analysis characterized in that the unit comprises a substrate formed of a material having properties of attenuating radiation and/or light and formed with a plurality of holes and adsorptive areas are respectively formed inside the above-mentioned a plurality of holes thereby forming a plurality of adsorptive areas.

(2) The unit for biochemical analysis characterized in that the unit comprises a substrate formed of a material having properties of attenuating radiation and/or light and formed with a plurality of holes, and adsorptive areas are respectively formed inside the above-mentioned a plurality of holes thereby forming a plurality of adsorptive areas, wherein a specific binding substances whose structure or characteristics is known is spotted on the plurality of adsorptive areas formed inside the above-mentioned a plurality of holes, and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with the specific binding substances so that the above-mentioned plurality of adsorptive areas are selectively labeled.

(3) The unit for biochemical analysis in accordance with (2) characterized in that the substance derived from a living organism is bound with specific binding substances by a reaction selected from a group consisting of hybridization, antigen-antibody reaction and receptor-ligand reaction.

(4) The unit for biochemical analysis in accordance with any one of (1) to (3) characterized in that the plurality of adsorptive areas are formed by charging an adsorptive material in the plurality of holes formed in the substrate.

(5) The unit for biochemical analysis in accordance with any one of (1) to (4) characterized in that each of the plurality of holes is formed as a through-hole.

(6) The unit for biochemical analysis in accordance with any one of (1) to (4) characterized in that each of the plurality of holes is formed as a reentrant.

(7) The unit for biochemical analysis in accordance with any one of (1) to (6) characterized in that the substrate is formed of a flexible material.

(8) The unit for biochemical analysis in accordance with any one of (1) to (7) characterized in that the substrate is formed with a gripping portion by which the substrate can be gripped.

(9) The unit for biochemical analysis characterized in that the unit comprises an adsorptive substrate formed of an adsorptive material and a perforated plate formed with a plurality of through-holes and formed of a material having properties of attenuating radiation and/or light, the perforated plate being closely contacted with at least one surface of the adsorptive substrate to form a plurality of adsorptive areas of the adsorptive substrate in the plurality of through-holes formed in the perforated plate.

(10) The unit for biochemical analysis in accordance with (9) characterized in that perforated plates are in close contact with the both surfaces of the adsorptive substrate.

(11) The unit for biochemical analysis in accordance with (9) or (10) characterized in that the perforated plate is formed with a gripping portion by which the perforated plate can be gripped.

(12) The unit for biochemical analysis in accordance with any one of (9) to (11) characterized in that a specific binding substance whose structure or characteristics is known is spotted on the plurality of adsorptive areas of the adsorptive substrate, and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with the specific binding substances so that the above-mentioned plurality of adsorptive areas are selectively labeled.

(13) The unit for biochemical analysis in accordance with any one of (1) to (12) characterized in that 10 or more holes are formed.

(14) The unit for biochemical analysis in accordance with (13) characterized in that 1,000 or more holes are formed.

(15) The unit for biochemical analysis in accordance with (14) characterized in that 10,000 or more holes are formed.

(16) The unit for biochemical analysis in accordance with any one of (1) to (15) characterized in that each of the plurality of holes has a size of less than 5 mm².

(17) The unit for biochemical analysis in accordance with (16) characterized in that each of the plurality of holes has a size of less than 1 mm².

(18) The unit for biochemical analysis in accordance with (17) characterized in that each of the plurality of holes has a size of less than 0.01 mm².

(19) The unit for biochemical analysis in accordance with any one of (1) to (18) characterized in that the plurality of holes are formed at a density of 10 or more per cm².

(20) The unit for biochemical analysis in accordance with (19) characterized in that the plurality of holes are formed at a density of 1,000 or more per cm².

(21) The unit for biochemical analysis in accordance with (20) characterized in that the plurality of holes are formed at a density of 10,000 or more per cm².

(22) The unit for biochemical analysis in accordance with any one of (1) to (21) characterized in that the material having properties of attenuating radiation and/or light has a property of reducing the energy of radiation and/or light to ⅕ or less when the radiation and/or light travels in the material by a distance equal to that between neighboring adsorptive areas.

(23) The unit for biochemical analysis in accordance with (22) characterized in that the material having properties of attenuating radiation and/or light has a property of reducing the energy of radiation and/or light to 1/10 or less when the radiation and/or light travels in the material by a distance equal to that between neighboring adsorptive areas.

(24) The unit for biochemical analysis in accordance with (23) characterized in that the material having properties of attenuating radiation and/or light has a property of reducing the energy of radiation and/or light to 1/100 or less when the radiation and/or light travels in the material by a distance equal to that between neighboring adsorptive areas.

(25) The unit for biochemical analysis in accordance with any one of (22) to (24) characterized in that the substrate is formed of a material selected from a group consisting of metal material, ceramic material and plastic material.

(26) The unit for biochemical analysis in accordance with any one of (22) to (24) characterized in that the perforated plate is formed of a material selected from a group consisting of metal material, ceramic material and plastic material.

(27) The unit for biochemical analysis in accordance with any one of (4) to (26) characterized in that the adsorptive material is comprised of a porous material.

(28) The unit for biochemical analysis in accordance with (27) characterized in that the porous material is comprised of a carbon material or a material capable of forming a membrane filter.

(29) The unit for biochemical analysis in accordance with any one of (4) to (26) characterized in that the adsorptive material is comprised of a fibrous material.

Furthermore, the present invention relates to a method for biochemical analysis wherein the unit for biochemical analysis of the present invention as mentioned in the present specification is used, and wherein a specific binding substance whose structure or characteristics is known is immobilized via a covalent bond in the adsorptive areas of the unit for biochemical analysis, and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with the specific binding substance, thereby detecting the above-mentioned labeled substance derived from a living organism.

The details of the method for biochemical analysis according to the present invention are described by JP Patent Publication (Kokai) No. 2002-355036A (2002), and all the contents given in JP Patent Publication (Kokai) No. 2002-355036A (2002) shall be incorporated into the present specification as a part of disclosure of the present specification. JP Patent Publication (Kokai) No. 2002-355036A (2002) specifically describes the biochemical analysis methods given in the following (30) to (62), and these methods can be similarly used in the present invention.

(30) A biochemical analysis method characterized in that the method comprises; preparing a unit for biochemical analysis by spotting a specific binding substance, which can specifically binds with a substance derived from a living organism and whose structure or characteristics is known, in a plurality of adsorptive areas, each of which is formed in a plurality of holes formed in a substrate formed of a material having properties of attenuating radiation, and specifically binding a substance derived from a living organism and labeled with a radioactive labeling substance with the specific binding substance, thereby selectively labeling said plurality of adsorptive areas; superposing the unit for biochemical analysis on an accumulative phosphor sheet in which a photostimulable phosphor layer is formed so that the photostimulable phosphor layer faces the plurality of adsorptive areas, thereby exposing the photostimulable phosphor layer to the radioactive labeling substance contained in the plurality of adsorptive areas; irradiating the photostimulable phosphor layer exposed to the radioactive labeling substance with an exciting light, thereby exciting photostimulable phosphor contained in the photostimulable phosphor layer; photoelectrically detecting stimulated emission released from the photostimulable phosphor contained in the photostimulable phosphor layer, thereby producing biochemical analysis data; and effecting biochemical analysis based on the biochemical analysis data.

(31) The biochemical analysis method in accordance with (30) characterized in that the plurality of adsorptive areas are formed by charging an adsorptive material in the plurality of holes formed in the substrate of the unit for biochemical analysis.

(32) The biochemical analysis method in accordance with (30) or (31) characterized in that a plurality of dot-like photostimulable phosphor layer areas are formed spaced-apart from each other in the accumulative phosphor sheet in approximately the same pattern as that of the plurality of holes formed in the substrate of the unit for biochemical analysis, and the unit for biochemical analysis and the accumulative phosphor sheet are superposed on each other so that each of the plurality of dot-like photostimulable phosphor layer areas faces one of the plurality of adsorptive areas in the plurality of holes formed in the substrate of the unit for biochemical analysis, thereby exposing the plurality of dot-like photostimulable phosphor layer areas of the accumulative phosphor sheet to the radioactive labeling substance contained in the plurality of adsorptive areas.

(33) The biochemical analysis method in accordance with any one of (30) to (32) characterized in that the substrate of the unit for biochemical analysis is formed of a material having properties of attenuating radiation and light, and the biochemical analysis is effected based on biochemical analysis data produced by the steps of preparing the unit for biochemical analysis by specifically binding a substance derived from a living organism and labeled with a fluorescent substance, in addition to a radioactive labeling substance, with the specific binding substance, thereby selectively labeling the plurality of adsorptive areas, irradiating the unit for biochemical analysis with an exciting light, thereby exciting the fluorescent substance, and photoelectrically detecting fluorescence released from the fluorescent substance.

(34) The biochemical analysis method in accordance with any one of (30) to (32) characterized in that the substrate of the unit for biochemical analysis is formed of a material having properties of attenuating radiation and light, and the biochemical analysis is effected based on biochemical analysis data produced by preparing the unit for biochemical analysis by specifically binding a substance derived from a living organism and labeled with a labeling substance which generates chemiluminescent emission upon contact with a chemiluminescent substrate, in addition to a radioactive labeling substance, with the specific binding substance, thereby selectively labeling the plurality of adsorptive areas, bringing the unit for biochemical analysis into contact with a chemiluminescent substrate, and photoelectrically detecting chemiluminescent emission released from the labeling substance.

(35) The biochemical analysis method in accordance with any one of (30) to (32) characterized in that the substrate of the unit for biochemical analysis is formed of a material having properties of attenuating radiation and light, and the biochemical analysis is effected based on biochemical analysis data produced by the steps of preparing the unit for biochemical analysis by specifically binding a substance derived from a living organism and labeled with, in addition to a radioactive labeling substance, a fluorescent substance and a labeling substance which generates chemiluminescent emission upon contact with a chemiluminescent substrate, with the specific binding substance, thereby selectively labeling the plurality of adsorptive areas, irradiating the unit for biochemical analysis with an exciting light to excite the fluorescent substance, and photoelectrically detecting fluorescence released from the fluorescent substance, while bringing the unit for biochemical analysis into contact with a chemiluminescent substrate, and photoelectrically detecting chemiluminescent emission released from the labeling substance.

(36) A biochemical analysis method characterized in that the method comprises the steps of; preparing a unit for biochemical analysis which comprises an adsorptive substrate formed of an adsorptive material and a perforated plate formed of a material having properties of attenuating radiation and formed with a plurality of through-holes, the perforated plate being closely contacted with at least one surface of the adsorptive substrate to form a plurality of adsorptive areas of the adsorptive substrate in the plurality of through-holes formed in the perforated plate, the plurality of adsorptive areas being selectively labeled with a radioactive labeling substance by spotting a specific binding substance, which can specifically bind with a substance derived from a living organism and whose structure or characteristics is known, in the plurality of adsorptive areas, and specifically binding the substance derived from a living organism and labeled with a radioactive labeling substance with the specific binding substance; superposing the unit for biochemical analysis and a accumulative phosphor sheet in which a photostimulable phosphor layer is formed via the perforated plate so that the photostimulable phosphor layer faces the plurality of adsorptive areas, thereby exposing the photostimulable phosphor layer to the radioactive labeling substance contained in the plurality of adsorptive areas; irradiating the photostimulable phosphor layer exposed to the radioactive labeling substance with an exciting light to excite photostimulable phosphor contained in the photostimulable phosphor layer; photoelectrically detecting stimulated emission released from the photostimulable phosphor contained in the photostimulable phosphor layer to produce biochemical analysis data; and effecting biochemical analysis based on the biochemical analysis data.

(37) The biochemical analysis method in accordance with (36) characterized in that perforated plates are closely contacted with both surfaces of the adsorptive substrate, thereby forming the unit for biochemical analysis, and the unit for biochemical analysis and the accumulative phosphor sheet are superposed via one of the perforated plates so that the photostimulable phosphor layer faces the plurality of adsorptive areas and thereby the photostimulable phosphor layer is exposed to a radioactive labeling substance contained in the plurality of adsorptive areas.

(38) The biochemical analysis method in accordance with (36) or (37) characterized in that a plurality of dot-like photostimulable phosphor layer areas are formed spaced-apart in the accumulative phosphor sheet in approximately the same pattern as that of the plurality of through-holes formed in the perforated plate, and the unit for biochemical analysis and the accumulative phosphor sheet are superposed on each other so that each of the plurality of dot-like photostimulable phosphor layer areas faces one of the plurality of adsorptive areas via one of the through-holes formed in the perforated plate, thereby the plurality of dot-like photostimulable phosphor layer areas are exposed to a radioactive labeling substance contained in the plurality of adsorptive areas.

(39) The biochemical analysis method in accordance with any one of (36) to (38) characterized in that the perforated plate is formed of a material having properties of attenuating radiation and light, and the biochemical analysis is effected based on biochemical analysis data produced by the steps of preparing the unit for biochemical analysis by specifically binding a substance derived from a living organism and labeled with a fluorescent substance, in addition to a radioactive labeling substance, with the specific binding substance, thereby selectively labeling the plurality of adsorptive areas, irradiating the unit for biochemical analysis with an exciting light through the plurality of the through-holes formed in the perforated plate, thereby exciting the fluorescent substance, and photoelectrically detecting fluorescence released from the fluorescent substance.

(40) The biochemical analysis method in accordance with any one of (36) to (38) characterized in that the perforated plate is formed of a material having properties of attenuating radiation and light, and the biochemical analysis is effected based on biochemical analysis data produced by the steps of preparing the unit for biochemical analysis by specifically binding a substance derived from a living organism and labeled with, in addition to a radioactive labeling substance, a labeling substance which generates chemiluminescent emission upon contact with a chemiluminescent substrate, with the specific binding substance, thereby selectively labeling the plurality of adsorptive areas, bringing the unit for biochemical analysis into contact with a chemiluminescent substrate through the plurality of the through-holes formed in the perforated plate, and photoelectrically detecting chemiluminescent emission released from the labeling substance.

(41) The biochemical analysis method in accordance with any one of (36) to (38) characterized in that the perforated plate is formed of a material having properties of attenuating radiation and light, and the biochemical analysis is effected based on biochemical analysis data produced by the steps of preparing the unit for biochemical analysis by specifically binding a substance derived from a living organism and labeled with, in addition to a radioactive labeling substance, a fluorescent substance and a labeling substance which generates chemiluminescent emission upon contact with a chemiluminescent substrate, with the specific binding substance, thereby selectively labeling the plurality of adsorptive areas, irradiating the unit for biochemical analysis with an exciting light through the plurality of the through-holes formed in the perforated plate to excite the fluorescent substance, and photoelectrically detecting fluorescence released from the fluorescent substance, while bringing the unit for biochemical analysis into contact with a chemiluminescent substrate through the plurality of the through-holes formed in the perforated plate, and photoelectrically detecting chemiluminescent emission released from the labeling substance.

(42) A biochemical analysis method characterized in that the method comprises preparing a unit for biochemical analysis by spotting a specific binding substance, which can specifically bind with a substance derived from a living organism and whose structure or characteristics is known, in a plurality of adsorptive areas formed in a plurality of holes formed in a substrate formed of a material having properties of attenuating light, and specifically binding a substance derived from a living organism and labeled with a fluorescent substance with the specific binding substance, thereby selectively labeling a plurality of adsorptive areas, irradiating the unit for biochemical analysis with an exciting light, thereby exciting the fluorescent substance, photoelectrically detecting fluorescence released from the fluorescent substance, thereby producing biochemical analysis data, and effecting biochemical analysis based on the biochemical analysis data.

(43) A biochemical analysis method characterized in that the method comprises preparing a unit for biochemical analysis by spotting a specific binding substance which can specifically bind with a substance derived from a living organism and whose structure or characteristics is known, in a plurality of adsorptive areas formed in a plurality of holes formed in a substrate formed of a material having properties of attenuating light, and specifically binding a substance derived from a living organism and labeled with a labeling substance capable of generating chemiluminescent emission upon contact with a chemiluminescent substrate with the specific binding substances, thereby selectively labeling the plurality of adsorptive areas, bringing the unit for biochemical analysis into contact with a chemiluminescent substrate, photoelectrically detecting chemiluminescent emission released from the labeling substance, thereby producing biochemical analysis data, and effecting biochemical analysis based on the biochemical analysis data.

(44) A biochemical analysis method characterized in that the method comprises preparing a unit for biochemical analysis by spotting a specific binding substance which can specifically bind with a substance derived from a living organism and whose structure or characteristics is known, in a plurality of adsorptive areas formed in a plurality of holes formed in a substrate formed of a material having properties of attenuating light, and specifically binding a substance derived from a living organism and labeled with a fluorescent substance and a labeling substance capable of generating chemiluminescent emission upon contact with a chemiluminescent substrate with the specific binding substances, thereby selectively labeling the plurality of adsorptive areas, irradiating the unit for biochemical analysis with an exciting light to excite the fluorescent substance, and photoelectrically detecting fluorescence released from the fluorescent substance, thereby producing biochemical analysis data, while bringing the unit for biochemical analysis into contact with a chemiluminescent substrate, photoelectrically detecting chemiluminescent emission released from the labeling substance, thereby producing biochemical analysis data, and effecting biochemical analysis based on the biochemical analysis data.

(45) The biochemical analysis method in accordance with any one of (42) to (44) characterized in that the plurality of adsorptive areas are formed by charging an adsorptive material in the plurality of holes formed in the substrate of the unit for biochemical analysis.

(46) A biochemical analysis method characterized in that the method comprises bringing an adsorptive substrate made of an adsorptive material and formed with a plurality of adsorptive areas by spotting thereon a specific binding substance which can specifically bind with a substance derived from a living organism and whose structure or characteristics is known, the plurality of the adsorptive areas being selectively labeled by specifically binding a substance derived from a living organism and labeled with a fluorescent substance with the specific binding substances contained in the plurality of adsorptive areas, into contact with a perforated plate formed of a material having properties of attenuating light and formed with a plurality of through-holes at positions corresponding to the plurality of adsorptive areas formed in the adsorptive substrate, irradiating the plurality of adsorptive areas formed in the adsorptive substrate through the plurality of through-holes formed in the perforated plate to excite the fluorescent substance, photoelectrically detecting fluorescence released from the fluorescent substance, thereby producing biochemical analysis data, and effecting biochemical analysis based on the biochemical analysis data.

(47) The biochemical analysis method in accordance with (46) characterized in that the unit for biochemical analysis is prepared by bringing perforated plates into close contact with both surfaces of the adsorptive substrate, and biochemical data are produced by irradiating the plurality of adsorptive areas formed in the adsorptive substrate with an exciting light through the plurality of through-holes formed in one of the perforated plates to excite a fluorescent substance and photoelectrically detecting fluorescence released from the fluorescent substance.

(48) A biochemical analysis method characterized in that the method comprises the steps of bringing an adsorptive substrate made of an adsorptive material and formed with a plurality of adsorptive areas by spotting thereon specific binding substances which can specifically bind with a substance derived from a living organism and whose structure or characteristics is known, the plurality of the adsorptive areas being selectively labeled by specifically binding a substance derived from a living organism and labeled with a labeling substance capable of generating chemiluminescent emission upon contact with a chemiluminescent substrate with the specific binding substance contained in the plurality of adsorptive areas, into close contact with a perforated plate formed of a material having properties of attenuating light and formed with a plurality of through-holes at positions corresponding to the plurality of adsorptive areas formed in the adsorptive substrate, bringing a chemiluminescent substrate into contact with the plurality of adsorptive areas formed in the adsorptive substrate through the plurality of through-holes formed in the perforated plate, photoelectrically detecting chemiluminescent emission released from the labeling substance, thereby producing biochemical analysis data, and effecting biochemical analysis based on the biochemical analysis data.

(49) The biochemical analysis method in accordance with (48) characterized in that the unit for biochemical analysis is prepared by bringing perforated plates into close contact with the both surfaces of the adsorptive substrate, and biochemical data are produced by bringing a chemiluminescent substrate into contact with the plurality of adsorptive areas formed in the adsorptive substrate through the plurality of through-holes formed in one of the perforated plates and photoelectrically detecting chemiluminescent emission released from the labeling substance.

(50) A biochemical analysis method characterized in that the method comprises bringing an adsorptive substrate made of an adsorptive material and formed with a plurality of adsorptive areas by spotting thereon a specific binding substance which can specifically bind with a substance derived from a living organism and whose structure or characteristics is known, the plurality of the adsorptive areas being selectively labeled by specifically binding a substance derived from a living organism and labeled with a fluorescent substance and a labeling substance capable of generating chemiluminescent emission upon contact with a chemiluminescent substrate with the specific binding substances contained in the plurality of adsorptive areas, into close contact with a perforated plate formed of a material having properties of attenuating light and formed with a plurality of through-holes at positions corresponding to the plurality of adsorptive areas formed in the adsorptive substrate, irradiating the plurality of adsorptive areas formed in the adsorptive substrate through the plurality of through-holes formed in the perforated plate to excite the fluorescent substance, and photoelectrically detecting fluorescence released from the fluorescent substance, thereby producing biochemical analysis data, while bringing a chemiluminescent substrate into contact with the plurality of adsorptive areas formed in the adsorptive substrate through the plurality of through-holes formed in the perforated plate, and photoelectrically detecting chemiluminescent emission released from the labeling substance, thereby producing biochemical analysis data, and effecting biochemical analysis based on the biochemical analysis data.

(51) The biochemical analysis method in accordance with (50) characterized in that the unit for biochemical analysis is prepared by bringing perforated plates into close contact with the both surfaces of the adsorptive substrate, and biochemical analysis is effected based on the biochemical analysis data which are produced by irradiating the plurality of adsorptive areas formed in the adsorptive substrate with an exciting light through the plurality of through-holes formed in one of the perforated plates to excite a fluorescent substance and photoelectrically detecting fluorescence released from the fluorescent substance and are also produced by bringing a chemiluminescent substrate into contact with the plurality of adsorptive areas formed in the adsorptive substrate through the plurality of through-holes formed in one of the perforated plates and photoelectrically detecting chemiluminescent emission released from the labeling substance.

(52) The biochemical analysis method in accordance with any one of (36) to (41) and (45) to

(50) characterized in that the specific binding substance is spotted through the plurality of through-holes formed in the perforated plate in the plurality of adsorptive areas formed in the adsorptive substrate.

(53) The biochemical analysis method in accordance with any one of (30) to (52) characterized in that 10 or more holes are formed.

(54) The biochemical analysis method in accordance with any one of (30) to (53) characterized in that each of the plurality of holes has a size of less than 5 mm².

(55) The biochemical analysis method in accordance with any one of (30) to (54) characterized in that the plurality of holes are formed at a density of 10 or more per cm².

(56) The biochemical analysis method in accordance with any one of (30) to (41) and (52) to

(55) characterized in that the material having properties of attenuating radiation has a property of reducing the energy of radiation to ⅕ or less when the radiation travels in the material by a distance equal to that between neighboring adsorptive areas.

(57) The biochemical analysis method in accordance with any one of (33) to (35) and (39) to

(55) characterized in that the material having properties of attenuating light has a property of reducing the energy of light to ⅕ or less when the light travels in the material by a distance equal to that between neighboring adsorptive areas.

(58) The biochemical analysis method in accordance with (56) or (57) characterized in that the substrate is formed of a material selected from a group consisting of metal material, ceramic material and plastic material.

(59) The biochemical analysis method in accordance with (56) or (57) characterized in that the perforated plate is formed of a material selected from a group consisting of metal material, ceramic material and plastic material.

(60) The biochemical analysis method in accordance with any one of (31) to (41) and (45) to

(59) characterized in that the adsorptive material is comprised of a porous material.

(61) The biochemical analysis method in accordance with (60) characterized in that the porous material is comprised of a carbon material or a material capable of forming a membrane filter.

(62) The biochemical analysis method in accordance with any one of (31) to (41) and (45) to

(59) characterized in that the adsorptive material is comprised of a fibrous material.

In the present invention, the materials for forming the substrate or perforated plate of the unit for biochemical analysis are not limited particularly as long as they have properties of attenuating radiation and/or light, and both inorganic materials and organic materials can be used, and metal material, ceramic material or plastic material is preferably used.

Examples of the inorganic materials which can be preferably used as materials for forming the substrate or perforated plate of the unit for biochemical analysis in the present invention and are capable of attenuating radiation include metals such as gold, silver, copper, zinc, aluminum, titanium, tantalum, chromium, iron, nickel, cobalt, lead, tin and selenium; alloys such as brass, stainless steel and bronze; silicon materials such as silicon, an amorphous silicon, glass, quartz, silicon carbide and silicon nitride; metal oxides, such as aluminum oxide, magnesium oxide and zirconium oxide; inorganic salts such as tungsten carbide, calcium carbonate, calcium sulfate, hydroxyapatite and gallium arsenide. These may have any structure of a single crystal structure, an amorphous structure or may be a polycrystal sintered material like ceramics.

As an organic material capable of attenuating radiation in the present invention, a polymer compound can be used preferably, and examples of the polymer compounds which can be preferably used as materials for forming the substrate or perforated plate of the unit for biochemical analysis in the present invention and are capable of attenuating radiation include polyolefms such as polyethylene and polypropylene; acrylic resin such as polymethyl methacrylate, butyl acrylate/methyl methacrylate copolymer; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate and polyethylene terephthalate; nylons such as nylon-6, nylon-6,6, nylon-4,10; polyimide; polysulfone; polyphenylene sulfide; silicon-resins such as polydiphenylsiloxane; phenol resin such as novolak; epoxy resin; polyurethane; polystyrene; butadiene styrene copolymer; polysaccharide such as cellulose, cellulose acetate, nitrocellulose, starch, calcium alginate, hydroxypropylmethylcellulose; chitin; chitosan; urushi (Japanese lacquer); polyamides such as gelatin, collagen and keratin, and copolymers of these polymer compounds etc. These materials may be composite materials, and if needed, they can also be filled with metal oxide particles, glass fiber, etc., and can be also blended with an organic material.

The ability of attenuating radiation generally increases as the specific gravity is larger and accordingly when the substrate or perforated plate of the unit for biochemical analysis is formed with a material having properties of attenuating radiation in the present invention, it is preferable to form them with a compound material or a composite material having a specific gravity of 1.0 g/cm³ or more, and it is especially preferable to form them with a compound material or a composite material having a specific gravity of 1.5 g/cm³ or more 23 g/cm³ or less.

Examples of the inorganic materials which can be preferably used as materials for forming the substrate or perforated plate of the unit for biochemical analysis in the present invention and are capable of attenuating light include metals such as gold, silver, copper, zinc, aluminum, titanium, tantalum, chromium, iron, nickel, cobalt, lead, tin and selenium; alloys such as brass, stainless steel and bronze; silicon materials such as silicon, an amorphous silicon, glass, quartz, silicon carbide and silicon nitride; metal oxides, such as aluminum oxide, magnesium oxide and zirconium oxide; inorganic salts such as tungsten carbide, calcium carbonate, calcium sulfate, hydroxyapatite and gallium arsenide. These may have any structure of a single crystal structure, an amorphous structure or may be a polycrystal sintered material like ceramics.

As an organic material capable of attenuating light in the present invention, a polymer compound can be used preferably, and examples of the polymer compounds which can be preferably used as materials for forming the substrate or perforated plate of the unit for biochemical analysis in the present invention and are capable of attenuating light include polyolefms such as polyethylene and polypropylene; acrylic resin such as polymethyl methacrylate, butyl acrylate/methyl methacrylate copolymer; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate and polyethylene terephthalate; nylons such as nylon-6, nylon-6,6, nylon-4,10; polyimide; polysulfone; polyphenylene sulfide; silicon-resins such as polydiphenylsiloxane; phenol resin such as novolak; epoxy resin; polyurethane; polystyrene; butadiene styrene copolymer; polysaccharide such as cellulose, cellulose acetate, nitrocellulose, starch, calcium alginate, hydroxypropylmethylcellulose; chitin; chitosan; urushi (Japanese lacquer); polyamides such as gelatin, collagen and keratin, and copolymers of these polymer compounds etc. These materials may be composite materials, and if needed, they can also be filled with metal oxide particles, glass fiber, etc., and can be also blended with an organic material.

The ability of attenuating light generally increases as the scattering and/or absorption of light (absorbance) is larger, and accordingly when the substrate or perforated plate of the unit for biochemical analysis is formed with a material having properties of attenuating light in the present invention, it is preferable that the absorbance per cm in thickness is 0.3 or more, and it is more preferable that the absorbance per cm in thickness is 1 or more. The absorbance can be determined by placing an integrating sphere immediately behind a plate-like member having a thickness of T cm, measuring an amount A of transmitted light at a wavelength of probe light or emission light used for measurement by a spectrophotometer, and calculating A/T.

In the present invention, a light scattering substance or a light absorbing substance may be added to the substrate or the perforated plate of the unit for biochemical analysis in order to improve the ability of attenuating light. Particles of a material different from a material forming the substrate or the perforated plate of the unit for biochemical analysis may be preferably used as a light scattering substance, and a pigment or dye may be preferably used as a light absorbing substance.

In a preferred embodiment of the present invention, the substrate of the unit for biochemical analysis is formed of a flexible material.

According to this preferred embodiment of the present invention, since the substrate of the unit for biochemical analysis is formed of a flexible material, the unit for biochemical analysis can be bent and be brought into contact with a hybridization solution, thereby hybridizing the specific binding substance with a substance derived from a living organism. Therefore, the specific binding substance and the substance derived from a living organism can be hybridized with each other in a desired manner using a small amount of a hybridization solution.

In a preferred embodiment of the present invention, the each of the holes is regularly formed in the substrate of the unit for biochemical analysis, or each of the holes is formed substantially in a circular shape or each of the holes is formed substantially in a rectangular shape.

In a preferred embodiment of the present invention, the substrate of the unit for biochemical analysis is formed with 10 or more holes, more preferably 50 or more holes, still more preferably 100 or more holes, still more preferably 1,000 or more holes, still more preferably 10,000 or more holes, still more preferably 100,000 or more holes.

In a preferred embodiment of the present invention, each of the plurality of holes formed in the substrate of the unit for biochemical analysis has a size of less than 5 mm², more preferably less than 1 mm², still more preferably less than 0.5 mm², still more preferably less than 0.1 mm², still more preferably less than 0.05 mm², still more preferably less than 0.01 mm².

In the present invention, the density of the holes formed in the substrate of the unit for biochemical analysis is determined depending upon the material of the substrate, the thickness of the substrate, the kind of electron beam released from a radioactive substance, the wavelength of fluorescence released from a fluorescent substance or the like.

In a preferred embodiment of the present invention, the plurality of holes are formed in the substrate of the unit for biochemical analysis at a density of 10 or more per cm², more preferably 50 or more per cm², still more preferably 1000 or more per cm², still more preferably 500 or more per cm ², still more preferably 1000 or more per cm², still more preferably 5000 or more per cm², still more preferably 10000 or more per cm².

In a preferred embodiment of the present invention, the each of the through-holes is regularly formed in the substrate of the unit for biochemical analysis, or each of the through-holes is formed substantially in a circular shape or each of the through-holes is formed substantially in a rectangular shape.

In a preferred embodiment of the present invention, the perforated plate of the unit for biochemical analysis is formed with 10 or more through-holes, more preferably 50 or more through-holes, still more preferably 100 or more through-holes, still more preferably 1000 or more through-holes, still more preferably 10000 or more through-holes, still more preferably 100000 or more through-holes.

In a preferred embodiment of the present invention, each of the plurality of through-holes formed in the perforated plate of the unit for biochemical analysis has a size of less than 5 mm , more preferably less than 1 mm², still more preferably less than 0.5 mm², still more preferably less than 0.1 mm², still more preferably less than 0.05 mm², still more preferably less than 0.01 mm².

In the present invention, the density of the through-holes formed in the perforated plate of the unit for biochemical analysis is determined depending upon the material of the perforated plate, the thickness of the perforated plate, the kind of electron beam released from a radioactive substance, the wavelength of fluorescence released from a fluorescent substance or the like.

In a preferred embodiment of the present invention, the plurality of through-holes are formed in the perforated plate of the unit for biochemical analysis at a density of 10 or more per cm², more preferably 50 or more per cm², still more preferably 100 or more per cm², still more preferably 500 or more per cm², still more preferably 1000 or more per cm², still more preferably 5000 or more per cm², still more preferably 10000 or more per cm².

In the present invention, a porous material or a fibrous material may be preferably used as the absorptive material for forming the adsorptive area. The adsorptive area may be formed by combining a porous material and a fibrous material.

In the present invention, a porous material for forming the adsorptive area may be any type of an organic material or an inorganic material and may be an organic/inorganic composite material.

In the present invention, an organic porous material used for forming the adsorptive area is not particularly limited, and a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, cellulose acetate, cellulose butyrte acetate; collagen; alginic acids such as alginic acid, calcium alginate, alginic acid/polylysine polyionic complex; polyolefms such as polyethylene, polypropylene; polyvinyl chloride; polyvinylidene chloride; polyfluoride such as polyvinylidene fluoride, polytetrafluoride; and copolymers or composite materials thereof.

In the present invention, an inorganic porous material used for forming the adsorptive area is not particularly limited. Preferred examples of inorganic porous materials include metals such as platinum, gold, iron, silver, nickel, aluminum and the like; metal oxides such as alumina, silica, titania, zeolite and the like; metal salts such as hydroxyapatite, calcium sulfate and the like; and composite materials thereof.

In the present invention, a fibrous material used for forming the adsorptive area is not particularly limited. Preferred examples of fibrous materials include nylons such as nylon-6, nylon-6,6, nylon-4,10; and cellulose derivatives such as nitrocellulose, cellulose acetate, cellulose butyrate acetate.

In the present invention, the adsorptive area may be formed using an oxidization process such as an electrolytic process, a plasma process, an arc discharge process and the like; a primer process using a silane coupling agent, titanium coupling agent and the like; and a surface process such as surface-active agent process and the like.

In the present invention, in the case where a plurality of dot-like photostimulable phosphor layer areas are formed in the support of the accumulative phosphor sheet, the plurality of dot-like photostimulable phosphor layer areas may be formed on the surface of the support or the plurality of dot-like photostimulable phosphor layer areas may be formed in a plurality of holes formed dot-like in the support.

In the present invention, in the case where a plurality of dot-like photostimulable phosphor layer areas are formed in the support of the accumulative phosphor sheet, the plurality of dot-like photostimulable phosphor layer areas are formed in the same pattern as that of the adsorptive areas formed in the unit for biochemical analysis.

In a preferred embodiment of the present invention, a plurality of through-holes are formed dot-like in the support of the accumulative phosphor sheet, and photostimulable phosphor layer areas are formed in the plurality of through-holes.

In a further preferred embodiment of the present invention, photostimulable phosphor layer areas are formed by charging photostimulable phosphor in the plurality of through-holes.

In another preferred embodiment of the present invention, a plurality of recesses are dot-like formed in the support of the photostimulable phosphor sheet and photostimulable phosphor layer areas are formed in the plurality of reentrant.

In a further preferred embodiment of the present invention, photostimulable phosphor layer areas are formed by charging photostimulable phosphor in the plurality of reentrant.

In a preferred embodiment of the present invention, a plurality of dot-like photostimulable phosphor layer areas are regularly formed in the accumulative phosphor sheet.

In the present invention, in the case where a plurality of dot-like photostimulable phosphor layer areas are formed in the support of the accumulative phosphor sheet, the material for forming the support of the accumulative phosphor sheet preferably has a property of attenuating radiation. The material capable of attenuating radiation is not particularly limited, and may be of any type of inorganic compound material or organic compound material. Preferred examples are metal material, ceramic material or plastic material.

In the present invention, illustrative examples of inorganic compound materials capable of attenuating radiation and preferably usable for forming the support of the accumulative phosphor sheet in the present invention include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxyapatite, gallium arsenide and the like. These may have any of a monocrystal structure, an amorphous structure, a polycrystal sintered structure such as ceramic or the like.

In the present invention, a high molecular compound is preferably used as an organic compound material capable of attenuating radiation. Examples of high molecular compounds and preferably usable for forming a support of the accumulative phosphor sheet in the present invention include polyolefms such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifuluoroethylene; polycarbonate; polyesters such as polyethylene iiaphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, cellulose acetate, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the ability of attenuating radiation generally increases as the specific gravity increases, the support of the accumulative phosphor sheet is preferably formed of a compound material or a composite material having specific gravity of 1.0 g/cm³ or more and more preferably formed of a compound material or a composite material having specific gravity of 1.5 g/cm³ to 23 g/cm³.

In a preferred embodiment of the present invention, a material capable of attenuating radiation has property of reducing the energy of radiation to ⅕ or less, more preferably 1/10 or less, still more preferably 1/50 or less, still more preferably 1/100 or less, still more preferably 1/500 or less, still more preferably 1/1000 or less, when the radiation travels in the material by the distance between neighboring dot-like photostimulable phosphor layer areas.

In the present invention, the photostimulable phosphor used in the present invention may be of any type insofar as it can accumulate radiation energy and can be excited by an electromagnetic wave to release the radiation energy accumulated therein in the form of light. It is preferred to use one which can be excited by a light of wave length region of visible light. Preferably employed photostimulable phosphors include alkaline earth metal fluorohalide phosphors (Ba1−x, M2+x) FX:yA (where M2+ is at least one alkaline earth metal selected from the group consisting of Mg, Ca, Sr, Zn and Cd; X is at least one element selected from the group consisting of Cl, Br and I, A is at least one trivalent metal element selected from the group consisting of Eu, Th, Ce, Tm, Dy, Pr, Ho, Nd, Yb and Er; x is 0≦x≦0.6 and y is 0≦y≦0.2) disclosed in U.S. Pat. No. 4,239,968, alkaline earth metal fluorohalide phosphors SrFX:Z (where X is at least one halogen selected from the group consisting of Cl, Br and I; Z is at least one Eu and Ce) disclosed in JP Patent Publication (Kokai) No. 2-276997A (1990), europium activated complex halide phosphors BaFX.xNaX′:aEu2+ (where each of X or X′ is at least one halogen selected from the group consisting of Cl, Br and I; x is 0<x≦2; and a is 0<a≦0.2) disclosed in JP Patent Publication (Kokai) No. 59-56479A (1984), cerium activated trivalent metal oxyhalide phosphors MOX:xCe (where M is at least one trivalent metal element selected from the group consisting of Pr, Nd, Pm, Sm, Eu, Tb, Dy, Ho, Er, Tm, Yb and Bi; X is at least one halogen selected from the group consisting of Br and I; and x is 0<x<0.1) disclosed in JP Patent Publication (Kokai) No. 58-69281A (1983), cerium activated rare earth oxyhalide phosphors LnOX:xCe (where Ln is at least one rare earth element selected from the group consisting of Y, La, Gd and Lu; X is at least one halogen selected from the group consisting of Cl, Br and I; and x is 0<x≦0.1) disclosed in U.S. Pat. No. 4,539,137, and europium activated complex halide phosphors MII FX.aMIX′.bM′IIX″2.cMIIIX′″3.xA:yEu2+ (where MII is at least one alkaline earth metal element selected from the group consisting of Ba, Sr and Ca; MI is at least one alkaline metal element selected from the group consisting of Li, Na, K, Rb and Cs; M′II is at least one divalent metal element selected from the group consisting of Be and Mg; MIII is at least one trivalent metal element selected from the group consisting of Al, Ga, In and Ti; A is at least one metal oxide; X is at least one halogen selected from the group consisting of Cl, Br and I; each of X′, X″ and X′″ is at least one halogen selected from the group consisting of F, Cl, Br and I; a is 0≦a≦2; b is 0≦b≦10-2; c is 0≦c≦10-2; a+b+c≧10-2; x is 0<x≦0.5; and y is 0<y≦0.2) disclosed in U.S. Pat. No. 4,962,047.

In a preferred embodiment of the present invention, specific binding substances may be spotted onto the adsorptive areas of a unit for biochemical analysis using a spotting device.

In a preferred embodiment of the present invention, a spotting device is provided with a base plate onto which a support on which specific binding substances are to be spotted is to be placed, a spotting head capable of spotting specific binding substances, and a sensor for detecting a reference position of the adsorptive area to which specific binding substances are to be spotted.

In a preferred embodiment of the present invention, a spotting device is further provided with a drive mechanism for at least one-dimensionally and intermittently moving the spotting head and the base plate relative to each other.

According to this preferred embodiment of the present invention, since a spotting device is provided with a drive mechanism for at least one-dimensionally and intermittently moving the spotting head and the base plate relative to each other, specific binding substances can be reliably spotted onto the adsorptive areas formed in a unit for biochemical analysis in at least one-dimension by using the sensor to detect the adsorptive areas of the unit for biochemical analysis placed on the base plate for spotting with specific binding substances, thereby determining the relative positional relationship between the spotting head of the spotting device and the base plate on which the unit for biochemical analysis is placed, and spotting specific binding substances from the spotting head, while operating the driving mechanism for at least one dimensionally and intermittently moving the spotting head and the base plate relative to each other.

In a further preferred embodiment of the present invention, the drive mechanism is adapted for at least one-dimensionally moving the spotting head and the base plate relative to each other at a constant pitch.

According to this further preferred embodiment of the present invention, since the drive mechanism is adapted for at least one-dimensionally moving the spotting head and the base plate relative to each other at a constant pitch, specific binding substances can be reliably spotted onto the adsorptive areas formed in a unit for biochemical analysis in at least one-dimension by using the sensor to detect the adsorptive areas of the unit for biochemical analysis placed on the base plate for spotting with specific binding substances, thereby determining the relative positional relationship between the spotting head of the spotting device and the base plate on which the unit for biochemical analysis is placed, and spotting specific binding substances from the spotting head, while operating the driving mechanism for at least one dimensionally moving the spotting head and the base plate relative to each other at a constant pitch.

In a preferred embodiment of the present invention, the drive mechanism is adapted for relatively and intermittently moving the spotting head and the base plate in two dimensions.

According to this preferred embodiment of the present invention, since the drive mechanism is adapted for relatively and intermittently moving the spotting head and the base plate in two dimensions, specific binding substances can be reliably spotted onto the adsorptive areas two-dimensionally formed in a unit for biochemical analysis by using the sensor to detect the adsorptive areas of the unit for biochemical analysis placed on the base plate for spotting with specific binding substances, thereby determining the relative positional relationship between the spotting head of the spotting device and the base plate on which the unit for biochemical analysis is placed, and spotting specific binding substances from the spotting head, while operating the driving mechanism for relatively and intermittently moving the spotting head and the base plate in two dimensions.

In a further preferred embodiment of the present invention, the drive mechanism is adapted for relatively moving the spotting head and the base plate at a constant pitch in two dimensions.

According to this further preferred embodiment of the present invention, since the drive mechanism is adapted for relatively moving the spotting head and the base at a constant pitch in two dimensions, specific binding substances can be reliably spotted onto the adsorptive areas two-dimensionally formed in a unit for biochemical analysis by using the sensor to detect the adsorptive areas of the unit for biochemical analysis placed on the base plate for spotting with specific binding substances, thereby determining the relative positional relationship between the spotting head of the spotting device and the base plate on which the unit for biochemical analysis is placed, and spotting specific binding substances from the spotting head, while operating the driving mechanism for relatively and intermittently moving the spotting head and the base plate at a constant pitch in two dimensions.

In a preferred embodiment of the present invention, at least two positioning members are formed in the base plate for positioning the unit for biochemical analysis.

According to this preferred embodiment of the present invention, since at least two positioning members are formed in the base plate for positioning a unit for biochemical analysis, it is possible to position the unit for biochemical analysis onto which specific binding substances are to be spotted at a predetermined position of the base plate and set it on the base plate.

In a further preferred embodiment of the present invention, each of the positioning members is constituted as a pin uprightly formed on the base plate.

According to this preferred embodiment of the present invention, since each of the positioning members is constituted as a pin uprightly formed on the base plate, it is possible to easily position the unit for biochemical analysis onto which specific binding substances are to be spotted at a predetermined position of the base plate and set it on the base plate by forming the unit for biochemical analysis with positioning through-holes corresponding to the pins.

In a preferred embodiment of the present invention, the spotting device is further provided with positional data calculating means for calculating positional data of the adsorptive areas of the unit for biochemical analysis onto which specific binding substances are to be spotted based on at least two reference positions of the unit for biochemical analysis detected by the sensor; a memory for storing the positional data of the adsorptive areas of the unit for biochemical analysis onto which specific binding substances are to be spotted calculated by the positional data calculating means; and position control means for controlling the drive mechanism in accordance with the positional data of the adsorptive areas of the unit for biochemical analysis onto which specific binding substances are to be spotted, which were stored in the memory.

According to this preferred embodiment of the present invention, since the spotting device is further provided with positional data calculating means for calculating positional data of the adsorptive areas of the unit for biochemical analysis onto which specific binding substances are to be spotted based on at least two references positions of the unit for biochemical analysis detected by the sensor, a memory for storing the positional data of the adsorptive areas of the unit for biochemical analysis onto which specific binding substances are to be spotted calculated by the positional data calculating means, and position control means for controlling the drive mechanism in accordance with the positional data of the adsorptive areas of the unit for biochemical analysis onto which specific binding substances are to be spotted, which were stored in the memory, it is possible to automatically spot specific binding substances onto a plurality of adsorptive areas spaced-apart and dot-like formed in the substrate.

JP Patent Publication (Kokai) No. 2002-355036A (2002) describes specific examples of the unit for biochemical analysis in FIGS. 1 to 24. The units for biochemical analysis similar to those in FIGS. 1 to 24, provided that the adsorptive area or adsorptive material should have covalently binding functional groups, can be used in the present invention.

The present invention will be described more specifically by the following examples although the present invention is not limited by these examples.

### EXAMPLES

### Comparative Example 1

### Manufacturing Process for Biochemical Analysis Unit (A) Using Non-charge Nylon-6,6

(1) A SUS304 sheet having a size of 80 mm×80 mm and a thickness of 100 μm is perforated by etching to form the total of 6400 fine holes composed of 10×10 holes as one unit so that each of the holes has a circular opening of a diameter of 0.2 mm with a hole pitch of 0.3 mm and a hole interval of 0.1 mm.

(2) A non-charge nylon filter (product of Millipore Corp.) is superposed on the substrate obtained in (1) and sent in between the press roll heated at 150° C. and a backup roll, and is pressed by pressure of 20 kgf/cm², thereby the nylon filter is pressed into the holes of the substrate to obtain a unit for biochemical analysis (A).

### Comparison Example 2

### Method of Immobilization of Oligo to Biochemical Analysis Unit (A)

5′-end aminated oligo (GFP-70mer-NH2, product of Sigma Genosys) is diluted to 50 μM with PBS, spotted on the adsorptive area by a spotter. After being baked at 80° C. for 20 minutes, it is irradiated with UV of 33 mJ/cm².

### Comparison Example 3

### Methods of Preparation of Digoxigenin Labeled GFP, Hybridization and Detection

(1) 500 ng of GFP-cRNA, 100 μM digoxigenin dUTP (stable in alkali condition, product of Roche A.G.), 100 μM dTTP, 500 μM dATP.dGTP.dCTP, Oligo-dT 12-18 primer (product of Invitrogen) and RNaseOUT (product of Invitrogen) are mixed to total of 20 μl. 1 μl of SuperScriptII reverse transcriptase (product of Invitrogen) is added thereto and reacted at 42° C. for 50 minutes. After the reaction is stopped by treating at 70° C. for 15 minutes, 1 μl of RNaseH (product of Invitrogen) is added and RNA is decomposed at 37° C. for 15 minutes. This is purified in ChromaSpinTE-30 (product of Clontech), and the digoxigenin labeled GFP is obtained.

(2) After 1, 10 and 100 pg of digoxigenin labeled GFP are heat-denatured, they are added to 4 ml of a hybridization buffer. A prehybridization buffer (4 ml) which has been kept warmed at 60° C. beforehand is circulated for 1 hour across the adsorptive area of the above-mentioned unit for biochemical analysis (A) (linear speed at 0.2 cm/sec). Then the above-mentioned hybridization buffer is similarly circulated at 60° C. for 18 hours across the adsorptive area. Next, the washing buffer 1 is circulated for 5 minutes twice, and further the washing buffer 2 is circulated for 5 minutes twice (each at 60° C.) to conduct washing. Then, a blocking buffer is filtered beforehand through Ultrafree having a pore size of 0.22 μm (product of Millipore Corp.), and is circulated for 10 minutes and the circulation is stopped for 50 minutes. All the procedures below are carried out at room temperature. An alkali phophatase labeled anti-digoxigenin antibody is filtered beforehand through Ultrafree having a pore size of 0.22 μm (product of Millipore Corp.), and 1/10000 volume thereof is added to a blocking buffer which was filtered beforehand through Ultrafree having a pore size of 0.22 μm (product of Millipore Corp.), and is circulated for 1 minute and the circulation is stopped for 60 minutes. Then, a chemilumi washing liquid is circulated for 15 minutes. This is repeated 3 times, and a reaction with CDP-Star (ready-to-use, product of Roche A. G.) which is a chemiluminescence substrate is conducted finally for 1 hour, and the amount of luminescence is detected by LAS1000 (product of Fuji Films Co., Ltd.).

### Example 1

### Process for Production of Biochemical Analysis Unit (B) Using BiodyneC (COOH-introduced type nylon-6,6 membrane)

A unit for covalent bond type biochemical analysis (B) is obtained as in Comparative Example 1 except that BiodyneC (COOH-introduced type nylon-6,6 membrane) is pressed into instead of a non-charge nylon filter.

### Example 2

### Method for Activation of the COOH of (B) Produced in the Example 1

The unit for biochemical analysis (B) produced in the Example 1 is placed in a bat containing an aqueous solution of 1M 1-ethyl-3-(3-dimethylaminopropyl)carbodiimide hydrochloride (abbreviated as EDC, from the catalog of WAKO Pure Chemicals, Co.) (water-soluble carbodiimide)/1M NHS (N-hydroxysuccinimide), and shaken at room temperature for 1 hour. After that, it is rinsed with ethanol and dried at room temperature to obtain a unit for biochemical analysis (C).

### Example 3

### Process for Production of Biochemical Analysis Unit (D) on which Oligonucleotides are Immobilized

5′-end aminated oligo (NH2-GFP<70mer>, product of Sigma Genosys) is diluted to 50 μM with PBS, spotted on the adsorptive area of (C) by a spotter. Reaction is conducted under a saturated salt solution vapor at room temperature for 1 hour. After it is rinsed with a 0.1% Tween20/TBS, it is moved to a bat containing 0.1N NaOH and is shaken at room temperature for 10 minutes. Washing with Milli-Q water for 5 minutes is repeated three times, and thus a unit for biochemical analysis (D) is obtained.

### Example 4

### Detection of the Digoxigenin Labeled GFP Using the Unit for Biochemical Analysis (D)

Hybridization of the digoxigenin labeled GFP and detection are performed as in the method of Comparative Example 3 by using the unit for biochemical analysis (D) produced in Example 3.

### Example 5

### Process for Production of Biochemical Analysis Unit (E) on which Bifunctional Spacers are Immobilized

The unit for biochemical analysis (C) produced in the Example 2 is immersed in a solution of 10 mM sulfo-KMUS (N-[κ-maleimidoundecanolyloxy]-dulgosccinimide ester, product of PIERCE) in PBS, and shaken at room temperature for 1 hour. After that, it is rinsed with ethanol and dried at room temperature to obtain a unit for biochemical analysis (E) having maleimide groups on the surface via a spacer of (CH2)10.

### Example 6

### Process for Production of Biochemical Analysis Unit (F) on which Oligonucleotides are Immobilized via the Spacer

5′-end thiolated oligo (SH-GFP<70mer>, product of Sigma Genosys) is diluted to 50 μM with PBS, spotted on the adsorptive area of (E) by a spotter. Reaction is conducted under a saturated salt solution vapor at room temperature for 1 hour. After it is immersed in a 2% mercaptoethanol solution in PBS buffer and shaken for 30 minutes, it is moved to a bat containing 0.1N NaOH and is shaken at room temperature for 10 minutes. Washing with Milli-Q water for 5 minutes is repeated three times, and thus a unit for biochemical analysis (F) is obtained.

### Example 7

### Detection of the Digoxigenin Labeled GFP Using the Unit for Biochemical Analysis (F)

Hybridization of the digoxigenin labeled GFP and detection are performed as in the method of Comparative Example 3 by using the unit for biochemical analysis (F) produced in Example 6.

The results of detection in Comparative Example 3, Example 4 and Example 7 are shown in the following Table 1. As shown in the results of Table 1, when an oligonucleotide end is covalently bound and immobilized to the adsorptive area (Example 4 and Example 7), relative signal intensity is markedly increased as compared with the immobilization by UV irradiation (Comparative Example 3). The reason for this is considered that since only the end of the oligonucleotide is immobilized by covalent bond immobilization, hybridization efficiency has been greatly improved as compared with the case of UV irradiation immobilization. The relative signal intensity of Example 7 using the unit for biochemical analysis (F) on which oligonucleotides are immobilized via the spacer of C10 alkyl is increased as compared with the results of Example 4, which shows that the effect of a spacer is also large.

TABLE 1
Comparative data with the comparative
example (relative signal intensity)

| | GFP 1 pg | GFP 10 pg | GFP 100 pg |
|---|---|---|---|
| Example 4(immobilization | 200 | 500 | 2000 |
| with covalent bond) | | | |
| Example 7(immobilization | 300 | 1000 | 5000 |
| with covalent bond via spacer) | | | |
| Comparative Example 3 | 100 | 100 | 100 |
| (UV irradiation) | | | |

### Example 8

### Introduction of Vinylsulfonyl Group to Nylon

A non-charge nylon filter (product of Millipore Corp.) is immersed in a 3 wt % solution of 1,2-bis(vinyl sulfonylacetamide)ethane in a borate buffer solution (pH 8), shaken at 25° C. for 120 minutes and washed with a sterilized distilled water. After dried at 40° C. for 30 minutes, vinyl sulfonylated nylon (G) is obtained.

### Example 9

### Process for Production of Covalent Bond Type Biochemical Analysis Unit (H)

A unit for covalent bond type biochemical analysis (H) is obtained as in Comparative Example 1 except that vinyl sulfonylated nylon (G) is pressed into instead of a non-charge nylon filter.

### Example 10

### Process for Production of Biochemical Analysis Unit (1) on which Oligo are Immobilized

5′-end aminated oligo (NH2-GFP<70mer>, product of Sigma Genosys) is diluted to 50 μM with PBS, spotted on the adsorptive area of (F) by a spotter. Reaction is conducted under a saturated salt solution vapor at room temperature for 1 hour. After shaken in a 0.5M glycine-borate buffer solution for 30 minutes, a unit for biochemical analysis (G) on which oligo are immobilized is obtained.

### Example 11

### Method for Preparation and Detection of Digoxigenin Labeled GFP

Hybridization and detection of the digoxigenin labeled GFP are conducted as in Comparative Example 3. The results are shown in the following Table 2. As shown in the results of Table 2, signal intensity is markedly increased as compared with the immobilization by UV irradiation. This is an effect resulted by introducing a covalently binding functional group by the treatment with a low molecular compound, 1,2-bis(vinyl sulfonylacetamide)ethane and immobilizing at the oligonucleotide ends.

TABLE 2
Comparative data with the comparative example
(relative signal intensity)

| | GFP 1 pg | GFP 10 pg | GFP 100 pg |
|---|---|---|---|
| Example 11 | 200 | 400 | 1000 |
| Comparative Example 3 | 100 | 100 | 100 |

The reagents used in the Examples are as follows:

Prehybridization and hybridization buffer: 0.5M church phosphate buffer, 1 mM EDTA, 7% SDS

Washing buffer 1: 40 mM church phosphate buffer, 1% SDS

Washing buffer 2: 0.1×SSC, 0.1% SDS

Chemilumi washing buffer (described in DIG Wash and Block buffer Set available from Roche)

Blocking buffer (described in DIG Wash and Block buffer Set available from Roche)

Detection buffer (described in DIG Wash and Block buffer Set available from Roche)

5′ end aminated oligo (GFP-70mer, product of Sigma Genosys) sequence:

| | |
|---|---|
| 5′-CAACAAAATACTCCAATTGGCGATGGCCCTGTCCT | (SEQ No: 1) |
| TTTACCAGACAACCATTACCTGTCCACACAATCTG-3′ | |

### Effect of the Invention

The present invention enables to provide a unit for biochemical analysis which is capable of carrying out strong and efficient immobilization of specific binding substances and can obtain specific and high signals by controlling the direction of the immobilized specific binding substances.

1. A unit for biochemical analysis wherein the unit comprises a substrate formed of a material having properties of attenuating radiation and/or light and formed with a plurality of holes, and adsorptive areas are respectively formed inside the plurality of holes, thereby forming a plurality of adsorptive areas, and wherein covalently binding functional groups are introduced onto the adsorptive areas.

2. A unit for biochemical analysis wherein the unit comprises an adsorptive substrate formed of an adsorptive material having covalently binding functional groups and a perforated plate formed with a plurality of through-holes and formed of a material having properties of attenuating radiation and/or light, said perforated plate being closely contacted with at least one surface of said adsorptive substrate to form a plurality of adsorptive areas of said adsorptive substrate in said plurality of through-holes formed in said perforated plate.

3. A unit for biochemical analysis wherein the unit comprises a substrate formed of a material having properties of attenuating radiation and/or light and formed with a plurality of holes, and adsorptive areas are respectively formed inside the plurality of holes thereby forming a plurality of adsorptive areas, and wherein a specific binding substance whose structure or characteristics is known is covalently bound on the adsorptive areas and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with said specific binding substance so that said plurality of adsorptive are selectively labeled.

4. The unit for biochemical analysis according to claim 3 wherein the specific binding substance whose structure or characteristics is known has a functional group.

5. The unit for biochemical analysis according to claim 3 wherein the specific binding substance having a functional group is selected from a group consisting of nucleic acids, proteins and peptides.

6. The unit for biochemical analysis according to claim 3 wherein the nucleic acids having a functional group are selected from a group consisting of nucleotide derivatives, peptide nucleic acids and LNA.

7. The unit for biochemical analysis according to claim 3 wherein the nucleotide derivatives having a functional group are oligonucleotides.

8. The unit for biochemical analysis according to claim 3 wherein the substance derived from a living organism is bound with said specific binding substance by a reaction selected from a group consisting of hybridization, antigen-antibody reaction and receptor-ligand reaction.

9. The unit for biochemical analysis according to claim 1 wherein the adsorptive areas hold the covalently binding functional groups via a spacer.

10. A method for biochemical analysis wherein the unit for biochemical analysis according to claim 1 is used, and wherein a specific binding substance whose structure or characteristics is known is covalently immobilized on the adsorptive areas of the unit for biochemical analysis, and a substance derived from a living organism and labeled with at least one kind of labeling substances selected from a group consisting of a radioactive labeling substance, a fluorescent substance and a labeling substance which generates chemiluminescent emission in contact with a chemiluminescent substrate is allowed to be specifically bound with the specific binding substance thereby detecting said labeled substance derived from a living organism.

11. The biochemical analysis method according to claim 10 wherein said substance derived from a living organism is specifically bound with said specific binding substance by a reaction selected from a group consisting of hybridization, antigen-antibody reaction and receptor-ligand reaction.

12. A method for producing a unit for biochemical analysis wherein the unit comprises a substrate formed of a material having properties of attenuating radiation and/or light and formed with a plurality of holes and adsorptive areas are respectively formed inside the plurality of holes thereby forming a plurality of adsorptive areas, which comprising a step of closely contacting a material having a covalently binding functional group with the substrate.

13. A method for manufacturing a unit for biochemical analysis wherein the unit comprises a substrate formed of a material having properties of attenuating radiation and/or light and formed with a plurality of holes and adsorptive areas are respectively formed inside the plurality of holes thereby forming a plurality of adsorptive areas, which comprises a step of introducing a covalently binding functional group into the adsorptive material closely contacted with the substrate.

14. The method for producing a unit for biochemical analysis according to claim 12 wherein the adsorptive material is a porous material.

15. A method for immobilizing a specific binding substance to the unit for biochemical analysis according to claim 1 which comprises a step of treating the adsorptive area where a functional group is held with an activating agent for improving reactivity.

16. The method for immobilizing specific binding substances according to claim 15 wherein, after a step of treating the adsorptive area where a functional group is held with an activating agent for improving reactivity, a specific binding substance having a functional groups is reacted and immobilized.

17. The method for immobilizing specific binding substances according to claim 15 wherein a spacer is held between the specific binding substances having a functional group and the adsorptive areas.


SEQUENCE LISTING (1 sequence)

SEQ ID NO: 1 - DNA, 70 bp, Artificial Sequence
  5′ end aminated oligo
caacaaaatactccaattggcgatggccctgtccttttaccagacaaccattacctgtccacacaatctg